(19) United States
(12) Patent Application Publication
(10) Pub. No.: US 2018/0304295 A1
(43) Pub. Date: Oct. 25, 2018

(54) SUNSCREEN MISTER DEVICE

(71) Applicant: Bradley O'Mara, Charleston, SC (US)

(72) Inventor: Bradley O'Mara, Charleston, SC (US)

(21) Appl. No.: 15/958,607

(22) Filed: Apr. 20, 2018

Related U.S. Application Data
(60) Provisional application No. 62/488,252, filed on Apr. 21, 2017.

(51) Int. Cl. B05B 15/62; B05B 16/80; G07F 13/08; B05B 12/02

(52) U.S. Cl. CPC B05B 15/62; B05B 16/80; A61M 35/00; B05B 12/02; A45D 2200/057; G07F 13/08

(57) ABSTRACT

An apparatus for applying sunscreen is provided The apparatus includes a housing, a means for adhering the housing to a vertical surface, one or more nozzles coupled to the housing, the one or more nozzles for dispensing sunscreen via a mist, a valve communicably coupled to the one or more nozzles, such that the valve regulates ingress of sunscreen to the one or more nozzles, a pressurized canister of sunscreen removably coupled to the valve via a fastener, such that sunscreen from the canister enters the valve, and a processor communicably coupled to the valve, the processor configured for starting a timer and activating the valve for a predefined period of time after the timer expires.

### CROSS-REFERENCE TO RELATED APPLICATIONS

This application claims priority to provisional application No. 62/488,252 filed Apr. 21, 2017 and entitled “Sunscreen Mister Device.” The subject matter of provisional application No. 62/488,252 is hereby incorporated by reference in its entirety.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### TECHNICAL FIELD

The technical field relates generally to the field of skincare and cosmetics, and more specifically, relates to devices for applying sunscreen.

### BACKGROUND

The global market for skin care products related to sun protection is currently about 8 billion. Much of this market is driven by the rising awareness over the importance of sun protection. Skin cancer is one of the major factors behind the importance of skin protection. One in five Americans will develop skin cancer in the course of a lifetime, and between 40 and 50 percent of Americans who live to age 65 will have a type of carcinoma at least once. Thus, sunscreen and other forms of skin protection have enjoyed increasing popularity in the U.S. and worldwide.

The currently available solutions for applying sunscreen to a person's body leaves a lot to be desired. The conventional method of using one's hands to apply sunscreen is inexact and can leave areas of the body untouched, which can lead to burned areas of skin. Furthermore, a person's hands are often left with the oily residue of the sunscreen, which can be annoying. Also, when a person is by himself or herself, there are areas of the body that cannot be reached, which can lead to a sunburn in those areas.

Other methods of applying sunscreen also do not account for important information about the surrounding situation, such as the amount of time the person has been in the sun, and the amount of time the current layer of sunscreen has been applied. Different types of sunscreen last for different amounts of time and sunscreen must be reapplied after a certain period of time has passed. There is currently no convenient way for said information to be available to a person receiving sun in the outdoors. Additional pertinent information includes the estimated time for re-applying sunscreen, the UV index in the person's area, the person's sun exposure, and the person's skin type. Again, at the current time there is no fitting solution for presenting this information in a usable way to a person while receiving sun in the outdoors.

Therefore, a need exists to overcome the problems with the prior art as discussed above, and particularly for a more efficient and user-friendly apparatus and method for applying sunscreen and tracking sun exposure.

### SUMMARY

An apparatus for applying sunscreen is provided. This Summary is provided to introduce a selection of disclosed concepts in a simplified form that are further described below in the Detailed Description including the drawings provided. This Summary is not intended to identify key features or essential features of the claimed subject matter. Nor is this Summary intended to be used to limit the claimed subject matter's scope.

In one embodiment, an apparatus for applying sunscreen is provided that solves the above-described problems. The apparatus comprises: a housing, a means for adhering the housing to a vertical surface, one or more nozzles coupled to the housing, the one or more nozzles for dispensing sunscreen via a mist, a valve communicably coupled to the one or more nozzles, such that the valve regulates ingress of sunscreen to the one or more nozzles, a pressurized canister of sunscreen removably coupled to the valve via a fastener, such that sunscreen from the canister enters the valve, and a processor communicably coupled to the valve, the processor configured for starting a timer and activating the valve for a predefined period of time after the timer expires.

### BRIEF DESCRIPTION OF THE DRAWINGS

The accompanying drawings, which are incorporated in and constitute a part of this disclosure, illustrate various example embodiments. In the drawings:

FIG. 1 is an illustration of a man using the apparatus for applying sunscreen and tracking sun exposure, according to an example embodiment;

FIG. 2A is an illustration of a front perspective view of the apparatus for applying sunscreen and tracking sun exposure, according to an example embodiment;

FIG. 2B is an illustration of a front view of the apparatus for applying sunscreen and tracking sun exposure, according to an example embodiment;

FIG. 2C is an illustration of a side view of the apparatus for applying sunscreen and tracking sun exposure, according to an example embodiment;

FIG. 2D is an illustration of a rear view of the apparatus for applying sunscreen and tracking sun exposure, according to an example embodiment;

FIG. 2E is an illustration of a top view of the apparatus for applying sunscreen and tracking sun exposure, according to an example embodiment;

FIG. 2F is an illustration of a bottom perspective view of the apparatus for applying sunscreen and tracking sun exposure, according to an example embodiment;

FIG. 2G is an illustration of an exploded top perspective view of the apparatus for applying sunscreen and tracking sun exposure, according to an example embodiment;

FIG. 2H is an illustration of a top perspective view of the cannister and top portions of the apparatus for applying sunscreen and tracking sun exposure, according to an example embodiment;

FIG. 3 is a block diagram showing the main components of the apparatus for applying sunscreen and tracking sun exposure, according to an example embodiment;

FIG. 4 is a flowchart showing the process of applying sunscreen using the apparatus for applying sunscreen and tracking sun exposure, according to an example embodiment; and

FIG. 5 is a block diagram showing a computing system useful for implementing the apparatus for applying sunscreen and tracking sun exposure, according to an example embodiment.

### DETAILED DESCRIPTION

The following detailed description refers to the accompanying drawings. While embodiments of the claimed subject matter may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the claimed subject matter. Instead, the proper scope of the claimed subject matter is defined by the appended claims.

The claimed subject matter improves over the prior art by providing an apparatus for quickly and easily applying sunscreen evenly over a user's entire body, even in difficult to reach areas. The claimed subject matter also improves over the prior art by providing an apparatus for applying sunscreen over a user's entire body, when the user is by himself and does not have another person to assist him. The claimed subject matter presents an inexpensively manufactured device that reduces or eliminates the incidence of sunburn on a user's skin. The claimed subject matter also accounts for the amount of time the individual has been in the sun, the amount of time the current layer of sunscreen has been applied, the estimated time for re-applying sunscreen, the UV index in the individual's area, the individual's sun exposure, and the individual's skin type, and the claimed subject matter presents the aforesaid information to the user in a usable way while the user is receiving sun in the outdoors.

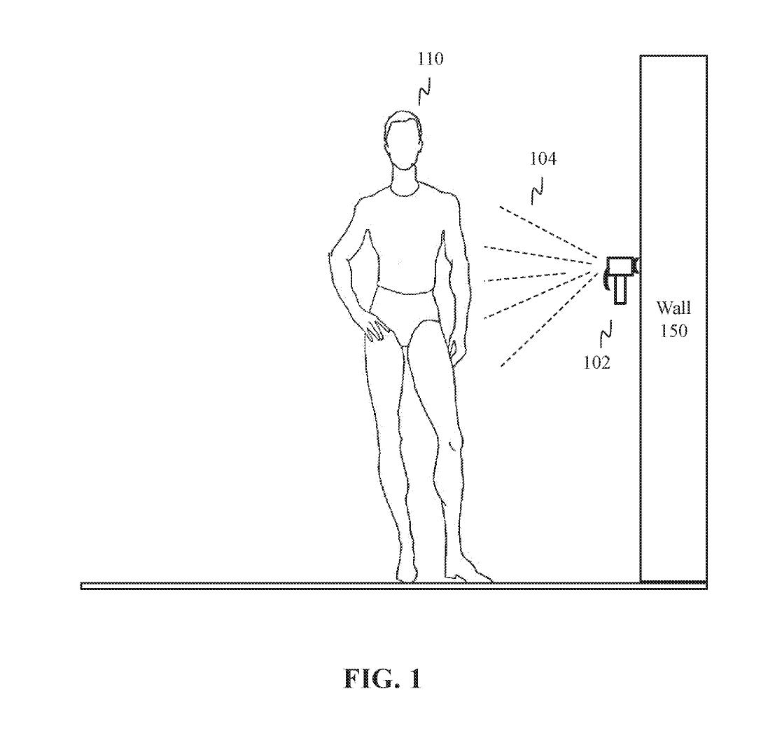

The apparatus for applying sunscreen and tracking sun exposure will now be described with reference to the figures. FIG. 1 is an illustration of a man 110 using the apparatus 102 for applying sunscreen 104 and tracking sun exposure, according to an example embodiment. FIG. 1 shows that the man 110 is by himself and would like to apply sunscreen to his skin in a uniform manner FIG. 1 shows that the man 110 may secure the apparatus 102 to a wall 150 at a height that is at or near the midpoint of the man's body. The man 110 may then turn his body, while remaining in the same place, as the apparatus 102 releases the sunscreen 104 as a spray or a mist that is expelled from the apparatus 102. As the man turns his body, the sunscreen 104 is applied evenly to his entire body.

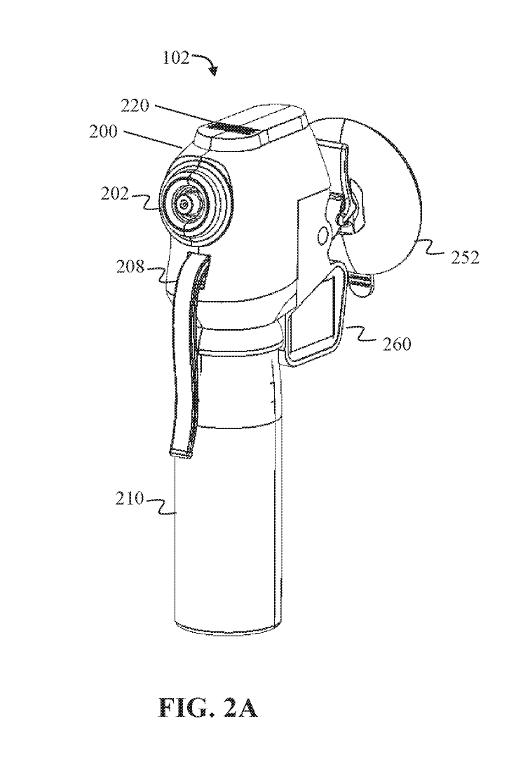

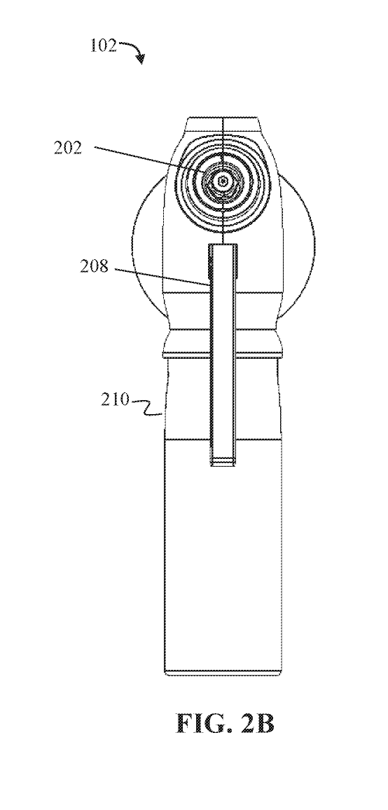

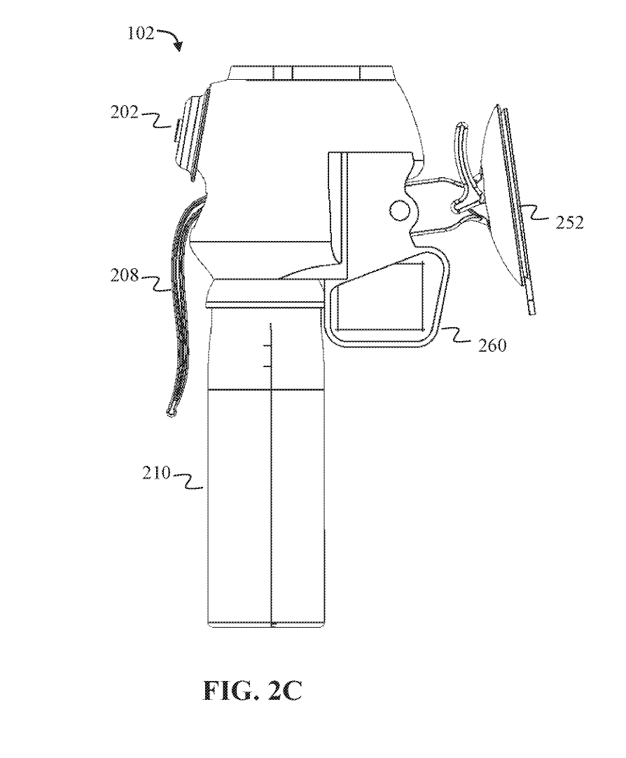

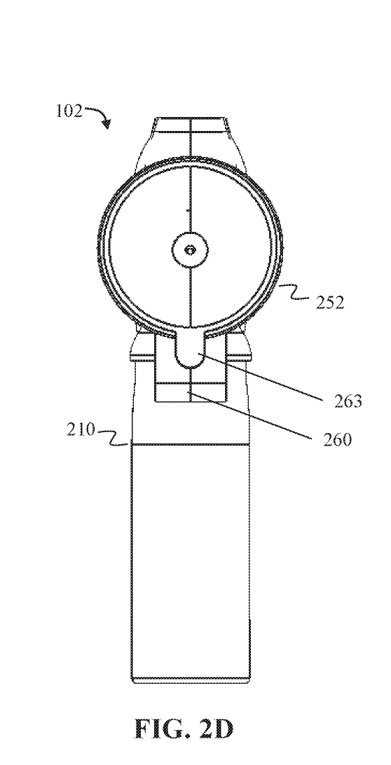

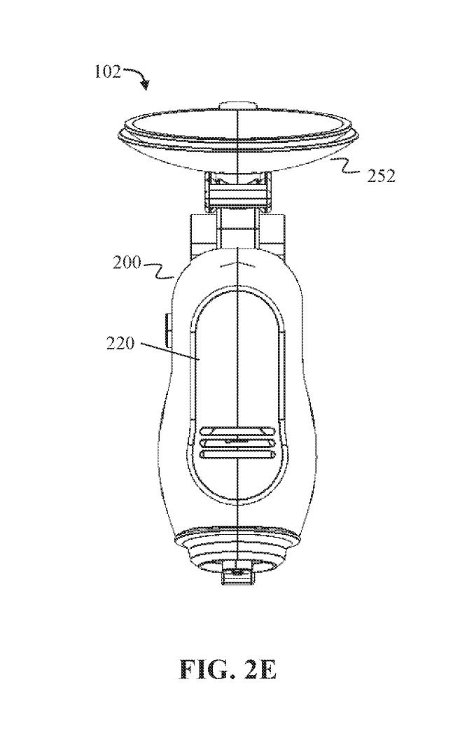

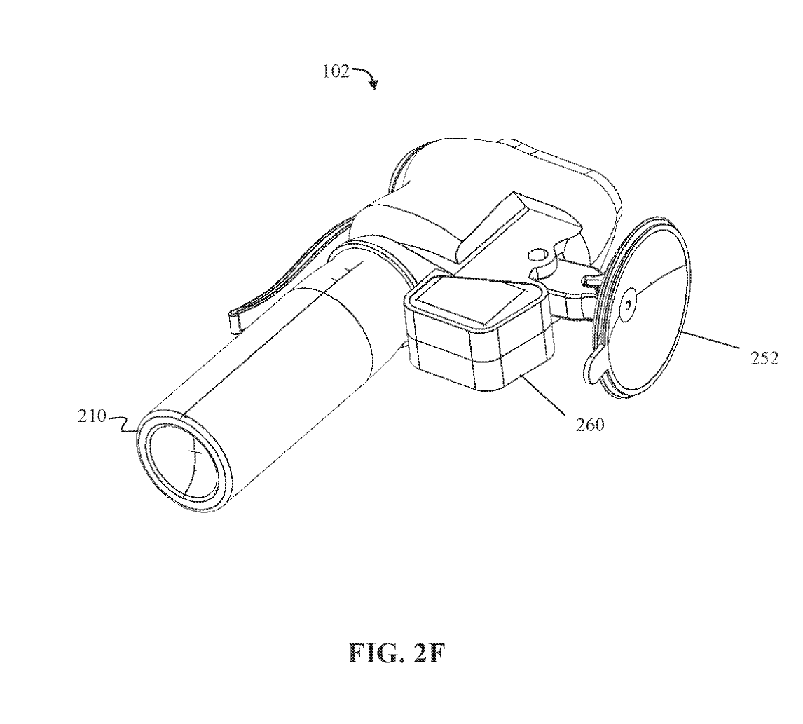

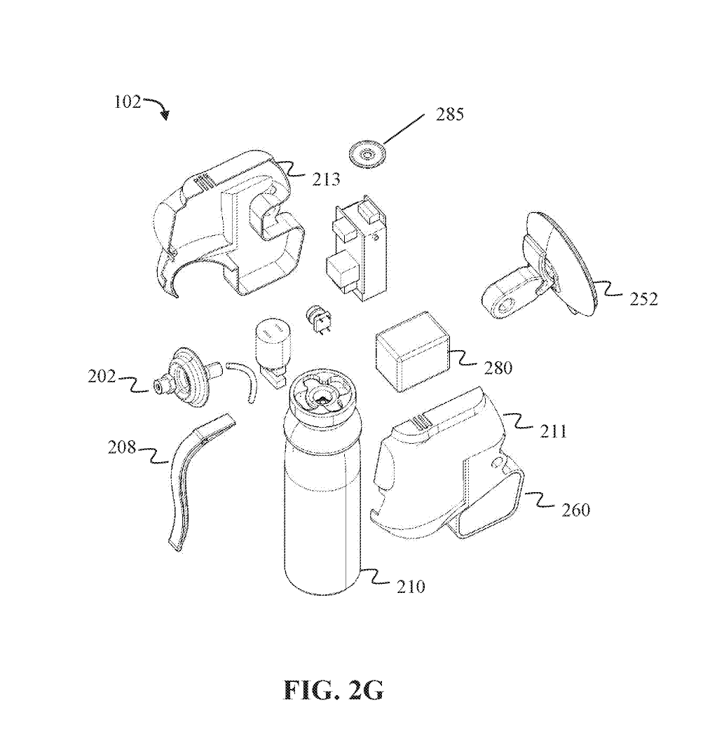

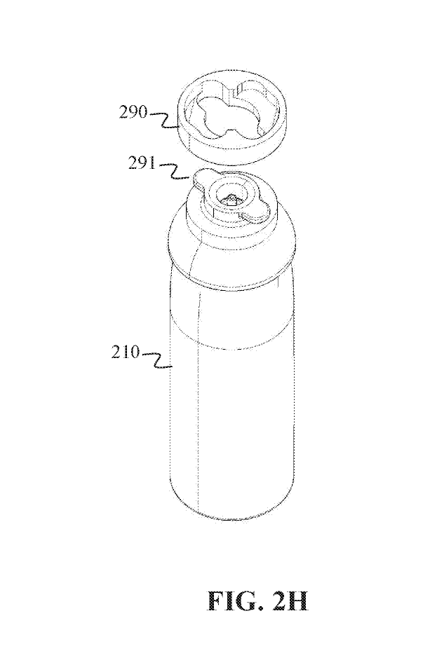

FIG. 2A is an illustration of a front perspective view of the apparatus 102 for applying sunscreen and tracking sun exposure, according to an example embodiment. FIG. 2A shows that the apparatus 102 includes a main housing 200 and a suction cup 252 coupled to a rear of the housing, such that the suction cup is configured to adhere to a surface, such as a wall or a window. The suction cup 252 may be configured to attach the apparatus 102 to a wall, a mirror or a glass panel at roughly the midpoint of the user's body or roughly chest height, such that the apparatus 102 can be used to apply sunscreen to a user's back or other areas that are difficult to reach. Attaching the apparatus 102 to a vertical surface allows a user 110 to avoid having to hold the apparatus while applying sunscreen to his body. Attaching the apparatus 102 to a vertical surface allows a user 110 to rotate his body while the apparatus remains stationary and applies sunscreen 104 to his body. The apparatus 102 may also include a handle 260 or flexible cord for holding the apparatus in one's hand or hanging the apparatus from a hook or other protrusion.

The apparatus 102 includes a pressurized canister 210 of sunscreen removably coupled to the main housing 200 via a fastener. The pressurized canister 210 may be screwed into the main housing 200 using threads on the canister and the housing. The pressurized canister 210 may alternatively be snapped (using a friction fit), or otherwise attached to, the main housing 200, using threads or similar protrusions on the cannister and housing. The canister 210 may be recyclable and may hold 8 oz. of volume. The canister 210 of sunscreen may be commercially available sunscreen.

The apparatus 102 also includes one or more nozzles 202 for dispensing the sunscreen 104 via a mist, wherein the one or more nozzles are communicably coupled to the canister 210. That is, there may be one or more conduits or tubular elements that communicably couple the nozzles 202 to the canister 210. A valve is communicably coupled to the canister 210 such that upon activation of the valve, pressure in the cannister drives sunscreen from the cannister through the conduits or tubular elements and out of the one or more nozzles 202 as a mist, so as to be applied to the individual's body. Each nozzle 202 may cover an 18-inch by 18-inch area with mist.

The apparatus 102 also includes a handle or lever 208 for dispensing the sunscreen 104 via a mist, wherein the handle or lever is communicably coupled to the valve. That is, the valve is communicably coupled to the handle or lever 208 such that upon pressing the handle or lever, the valve is activated and pressure in the cannister drives sunscreen from the cannister through the conduits or tubular elements and out of the one or more nozzles 202 as a mist, so as to be applied to the individual's body.

FIG. 2A also shows that the apparatus 102 includes a speaker 220 for playing audio that provides information to the user, as defined more fully below. The speaker 220 may also include a microphone for receiving audio commands from the user, such that the apparatus 102 may be voice activated.

FIG. 2B is an illustration of a front view of the apparatus 102 for applying sunscreen and tracking sun exposure, according to an example embodiment. FIG. 2B shows that the apparatus 102 includes a forward-facing handle or lever 208 for dispensing the sunscreen 104 via a mist, such that the handle or lever is located on the same side of the apparatus, and faces the same direction as, the one or more nozzles 202. This feature facilitates the utilization of the forward-facing handle or lever 208 for dispensing the sunscreen 104.

FIG. 2C is an illustration of a side view of the apparatus for applying sunscreen and tracking sun exposure, according to an example embodiment. FIG. 2C shows that the apparatus 102 may include one or more nozzles 202 that are pointing slightly upwards for properly dispensing the sunscreen 104 via a mist. This feature facilitates the uniform application of the sunscreen 104 on the user's body when the user is positioned in front of the apparatus 102, as shown in FIG. 1. In another embodiment, the one or more nozzles may point in other directions for properly dispensing the sunscreen 104 via a mist.

FIG. 2D is an illustration of a rear view of the apparatus 102 for applying sunscreen and tracking sun exposure, according to an example embodiment. FIG. 2D shows that apparatus 102 includes a suction cup 252 coupled to a rear of the housing 202, such that the suction cup is configured to adhere to a surface, such as a wall or a window. The suction cup 252 may be configured to attach the apparatus 102 to a wall, for example, and further includes a tab 263 that may be used to facilitate removing the suction cup from a surface, after the suction cup has been applied to the surface. In another embodiment, housing 200 may be adhered to a surface in other ways, such as using an adhesive, attachment using a hook, attachment using hook and loop fabric and attachment using a nail, screw or bolt. In this document, the term “means for adhering the housing to a vertical surface” includes all of the embodiments described above for adhering the housing 200 to a surface.

FIG. 2E is an illustration of a top view of the apparatus 102 for applying sunscreen and tracking sun exposure, according to an example embodiment. FIG. 2E shows that the apparatus 102 includes an upwards facing speaker 220 for playing audio that provides information to the user, as defined more fully below. This feature optimizes the level of volume of the sounds made by the speaker when the user is positioned in front of the apparatus 102, as shown in FIG. 1.

FIG. 2F is an illustration of a bottom perspective view of the apparatus 102 for applying sunscreen and tracking sun exposure, according to an example embodiment. The apparatus 102 includes the pressurized canister 210 of sunscreen removably coupled to the bottom portion of the main housing 200 via a fastener. The pressurized canister 210 may be screwed into the main housing 200 using threads on the canister and the housing.

FIG. 2G is an illustration of an exploded, or disassembled, top perspective view of the apparatus 102 for applying sunscreen and tracking sun exposure, according to an example embodiment. FIG. 2G shows that the housing 200 may comprise two halves 211 and 213 that come together to form the housing 200. FIG. 2G further shows the handle 260 and the suction cup 252. FIG. 2G also shows the lever 208, the cannister 210 and the nozzle 202. FIG. 2G further shows the diaphragm portion 285 of the speaker 220, and an element 280 that houses a processor, which is described more fully below.

FIG. 2H is an illustration of a top perspective view of the cannister 210 and top portions of the apparatus 102 for applying sunscreen and tracking sun exposure, according to an example embodiment. FIG. 2H shows that the top portion of the cannister 210 includes a fitting 291 that comprises a disc shaped element with a central orifice and two flanges that extend to either side of the disc shaped element. The fitting 291 is a separate piece that is attached to the cannister 210 such that the stem in the cannister extends through the orifice. The fitting 291 may be attached to the cannister 210 using a snap fitting or a screwed fitting using threads on the fitting and the cannister.

FIG. 2H also shows that the top portion of the cannister 210 includes a fitting 290 that comprises a disc shaped element with a central orifice that emulates the size and shape of the fitting 291, such that the fitting 291 can be inserted into and through the orifice in fitting 290. Fitting 290 also includes a cavity on its distal end that allows the fitting 291 to be rotated counterclockwise after fitting 291 has been inserted into and through the orifice in fitting 290. When rotated counterclockwise, the fitting 291 is removably secured to the fitting 290.

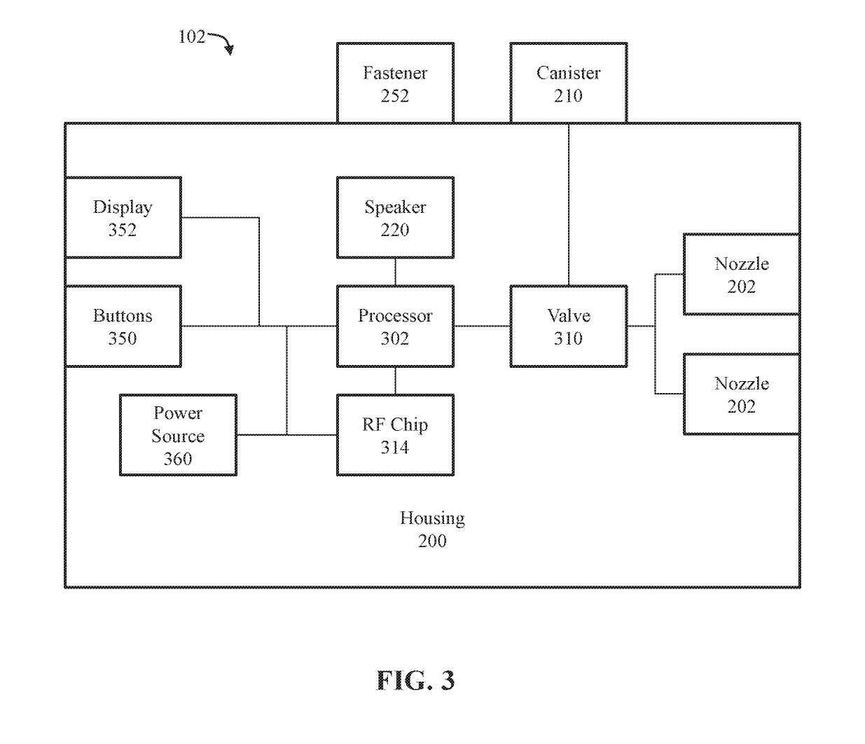

FIG. 3 is a block diagram showing the main components of the apparatus 102 for applying sunscreen and tracking sun exposure, according to an example embodiment. The apparatus includes a processor 302 that is communicably coupled with a display 352, buttons(s) 350, a power source 360, the speaker 220, radio frequency (RF) chip 314, and valve 310.

The apparatus 102 includes a processor 302 that, upon receiving a command, starts a timer and activates the valve 310 for a predefined period of time. The processor 310 may also detect the pressing of a push button 350, and then starts the timer. Once the timer expires, the processor 310 sends a signal to the valve 310 which opens to allow gas from the cannister 210 to drive sunscreen from the cannister through the conduits or tubular elements and out of the one or more nozzles 202 as a mist. After a predefined period of time, the processor 302 may send another signal to the valve 310, which closes the valve and stops the flow of sunscreen.

The apparatus 102 may also include a display 352 (such as an LCD display) for displaying time data, such as a current status of the timer. The display 352 may display, for example, a second countdown that indicates how many seconds until the mist begins or counts down the number of second left during misting of sunscreen. The apparatus 102 may also include a speaker 220 for playing audio received via the RF chip 314, otherwise known as a Bluetooth speaker. The apparatus 102 may also include a rechargeable battery (i.e., power source 360) for powering the processor, the speaker, the valve, and the display. The battery may be rechargeable via a USB connection. The apparatus 102 may also include an on/off button for turning the apparatus on and off.

The timer may also initiate an audio spoken timer using the speaker 220. The speaker 220 may countdown, for example, “5-4-3-2-1” until the sunscreen mist begins. The speaker 220 may also countdown, for example, “5-4-3-2-1” until the misting ends. The speaker 220 may also indicate when the misting is complete.

The Bluetooth connection for the speaker is the RF chip 314 and can also be used by the apparatus 102 to received commands from a mobile application on a mobile device. The mobile application may be used to turn the apparatus 102 on/off, track the time when sunscreen was applied, calculate when the sunscreen should be reapplied, determine the UV index for the user's global location (using location services on the mobile device), initiate an alarm to indicate when sunscreen should be reapplied, and track the user's activity, which may then be posted to social media. The mobile application may also be used to determine the user's skin type by taking a photo of the user's skin. The mobile application may also include an online ordering tool for ordering additional sunscreen, the sunscreen container and/or the gas canister.

Lastly, FIG. 3 shows that the apparatus 102 includes a fastener 252 which corresponds to the suction cup 252 coupled to a rear of the housing 200, such that the suction cup is configured to adhere to a surface, such as a wall or a window.

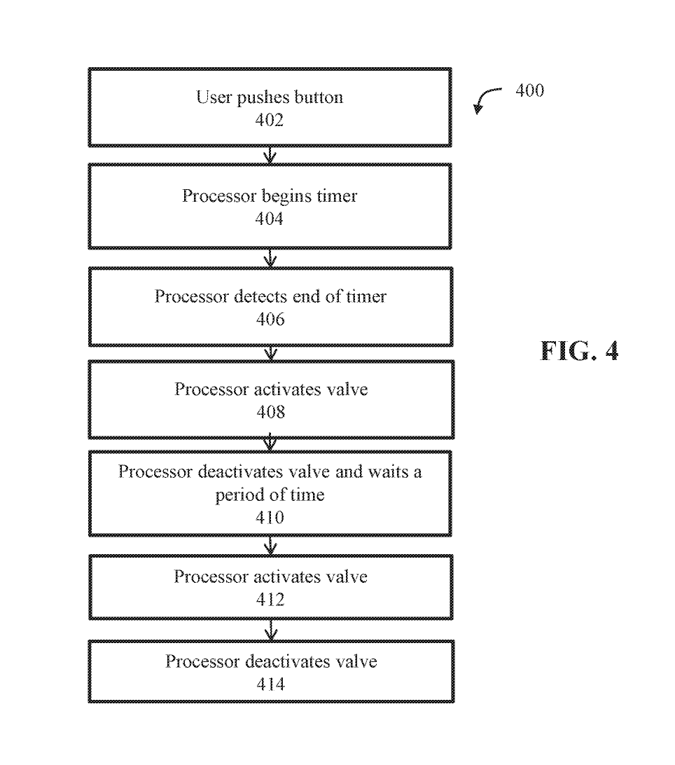

FIG. 4 is a flowchart showing the process 400 of applying sunscreen using the apparatus 102 for applying sunscreen and tracking sun exposure, according to an example embodiment. The following process 400 describes the process of applying sunscreen and tracking sun exposure using apparatus 102. Prior to the first step of the process 400, the user has secured the apparatus 102 to a surface, such as a wall, as in FIG. 1. The process 400 of applying sunscreen and tracking sun exposure begins with the user pushing a button 350 in step 402. Alternatively, the process 400 of applying sunscreen and tracking sun exposure begins with the user initiating the process via a mobile application in step 402, wherein the mobile application communicates with the apparatus 102 via RF chip 314. In step 404, the processor 302 beings a timer, such as a 5-second timer. In this step, the apparatus 102 may countdown the timer audibly via the speaker 220.

In step 406, the processor detects that the timer has expired and in step 408, the processor activates the valve 310, such that pressure in the cannister 210 drives sunscreen from the cannister through the conduits or tubular elements and out of the one or more nozzles 202 as a mist, so as to be applied to the individual's body. Step 408 may last for a predetermined time that is determined, or kept track of, by the processor. In step 410, the processor deactivates the valve 310 and sunscreen no longer exits the apparatus 102. Next, a predetermined time, which is determined, or kept track of, by the processor, is allowed to pass. This allows the user to turn around or readjust his body to allow for the sunscreen to be applied to a different part of his body. The apparatus 102 may countdown a timer audibly via the speaker 220. Then, in step 412, the processor activates the valve 310, such that pressure in the cannister 210 drives sunscreen from the cannister through the conduits or tubular elements and out of the one or more nozzles 202 as a mist, so as to be applied to the individual's body. Step 412 may last for a predetermined time that is determined, or kept track of, by the processor. In step 414, the processor deactivates the valve 310 and sunscreen no longer exits the apparatus 102.

In one embodiment, any of the components of the apparatus 102 for applying sunscreen and tracking sun exposure described above may comprise plastic, polyethylene, PVC, aluminum, steel, tin, copper, brass, iron, bronze, nickel, zinc, and any metal alloys. In another embodiment, the apparatus 102 may be waterproof, water resistant, shock resistant, heat resistant, cold resistant and drop resistant.

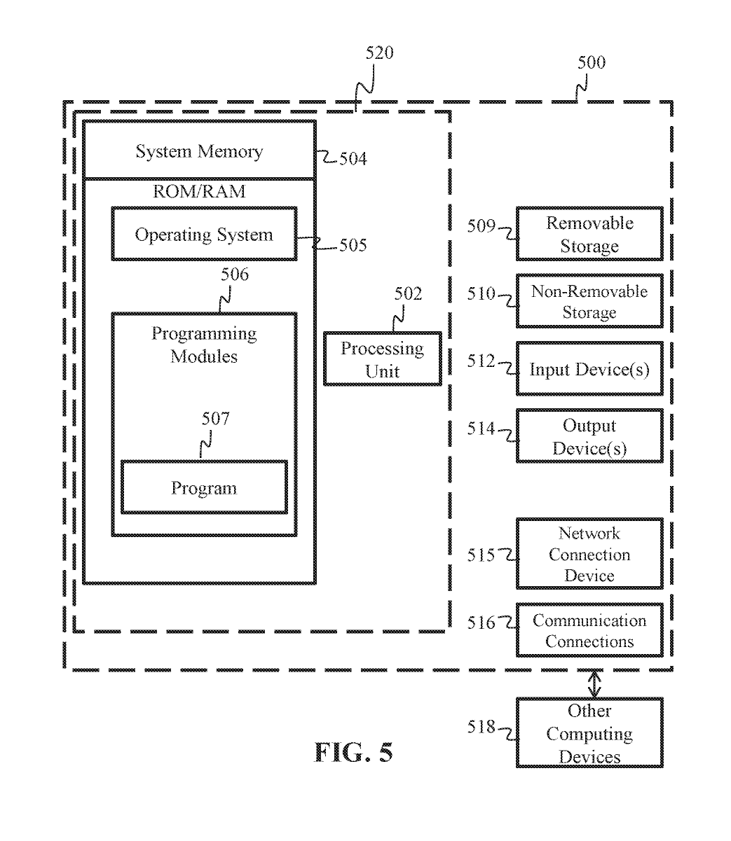

FIG. 5 is a block diagram showing a computing system useful for implementing the apparatus for applying sunscreen and tracking sun exposure, according to an example embodiment. Consistent with the embodiments described herein, the aforementioned actions performed by apparatus 102, and specifically processor 302, may be implemented in a computing device, such as the computing device 500 of FIG. 5. Any suitable combination of hardware, software, or firmware may be used to implement the computing device 500. The aforementioned system, device, and processors are examples and other systems, devices, and processors may comprise the aforementioned computing device. Furthermore, computing device 500 may comprise an operating environment for the methods shown in FIG. 4 above.

With reference to FIG. 5, a system consistent with one embodiment may include a plurality of computing devices, such as computing device 500. In a basic configuration, computing device 500 may include at least one processing unit 502 and a system memory 504. Depending on the configuration and type of computing device, system memory 504 may comprise, but is not limited to, volatile (e.g. random access memory (RAM)), non-volatile (e.g. read-only memory (ROM)), flash memory, or any combination or memory. System memory 504 may include operating system 505, one or more programming modules 506 (such as program module 507). Operating system 505, for example, may be suitable for controlling computing device 500's operation. In one embodiment, programming modules 506 may include, for example, a program module 507. Furthermore, embodiments described herein may be practiced in conjunction with a graphics library, other operating systems, or any other application program and is not limited to any particular application or system. This basic configuration is illustrated in FIG. 5 by those components within a dashed line 520.

Computing device 500 may have additional features or functionality. For example, computing device 500 may also include additional data storage devices (removable and/or non-removable) such as, for example, magnetic disks, optical disks, or tape. Such additional storage is illustrated in FIG. 5 by a removable storage 509 and a non-removable storage 510. Computer storage media may include volatile and nonvolatile, removable and non-removable media implemented in any method or technology for storage of information, such as computer readable instructions, data structures, program modules, or other data. System memory 504, removable storage 509, and non-removable storage 510 are all computer storage media examples (i.e., memory storage). Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 500. Any such computer storage media may be part of device 500. Computing device 500 may also have input device(s) 512 such as a keyboard, a mouse, a pen, a sound input device, a camera, a touch input device, etc. Output device(s) 514 such as a display, speakers, a printer, etc. may also be included. The aforementioned devices are only examples, and other devices may be added or substituted.

Computing device 500 may also contain a network connection device 515 that may allow device 500 to communicate with other computing devices 518, such as over a network in a distributed computing environment, for example, an intranet or the Internet. Device 515 may be a wired or wireless network interface controller, a network interface card, a network interface device, a network adapter or a LAN adapter. Device 515 allows for a communication connection 516 for communicating with other computing devices 518. Communication connection 516 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both computer storage media and communication media.

As stated above, a number of program modules and data files may be stored in system memory 504, including operating system 505. While executing on processing unit 502, programming modules 506 may perform processes including, for example, one or more of the methods shown in FIG. 4 above. The aforementioned processes are examples, and processing unit 502 may perform other processes. Other programming modules that may be used in accordance with embodiments described herein may include electronic mail and contacts applications, word processing applications, spreadsheet applications, database applications, slide presentation applications, drawing or computer-aided application programs, etc.

Generally, consistent with embodiments described herein, program modules may include routines, programs, components, data structures, and other types of structures that may perform particular tasks or that may implement particular abstract data types. Moreover, embodiments described herein may be practiced with other computer system configurations, including hand-held devices, multiprocessor systems, microprocessor-based or programmable consumer electronics, minicomputers, mainframe computers, and the like. Embodiments described herein may also be practiced in distributed computing environments where tasks are performed by remote processing devices that are linked through a communications network. In a distributed computing environment, program modules may be located in both local and remote memory storage devices.

Furthermore, embodiments described herein may be practiced in an electrical circuit comprising discrete electronic elements, packaged or integrated electronic chips containing logic gates, a circuit utilizing a microprocessor, or on a single chip (such as a System on Chip) containing electronic elements or microprocessors. Embodiments described herein may also be practiced using other technologies capable of performing logical operations such as, for example, AND, OR, and NOT, including but not limited to mechanical, optical, fluidic, and quantum technologies. In addition, embodiments described herein may be practiced within a general purpose computer or in any other circuits or systems.

Embodiments described herein, for example, are described above with reference to block diagrams and/or operational illustrations of methods, systems, and computer program products according to said embodiments. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments have been described, other embodiments may exist. Furthermore, although embodiments have been described as being associated with data stored in memory and other storage mediums, data can also be stored on or read from other types of computer-readable media, such as secondary storage devices, like hard disks, floppy disks, or a CD-ROM, or other forms of RAM or ROM. Further, the disclosed methods' stages may be modified in any manner, including by reordering stages and/or inserting or deleting stages, without departing from the embodiments described herein.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. An apparatus for applying sunscreen, the apparatus comprising:
(a) a housing;
(b) means for adhering the housing to a vertical surface;
(c) one or more nozzles coupled to the housing, the one or more nozzles for dispensing sunscreen via a mist;
(d) a valve communicably coupled to the one or more nozzles, such that the valve regulates ingress of sunscreen to the one or more nozzles;
(e) a pressurized canister of sunscreen removably coupled to the valve via a fastener, such that sunscreen from the canister enters the valve; and
(f) a processor communicably coupled to the valve, the processor configured for starting a timer and activating the valve for a predefined period of time after the timer expires.

2. The apparatus of claim 1, further comprising a push button coupled to the housing, the push button configured for activating the timer.

3. The apparatus of claim 2, further comprising a rechargeable battery configured for powering the processor and the valve, wherein the rechargeable battery is recharged via one or more solar panels.

4. The apparatus of claim 3, further comprising a display coupled to the housing and communicably coupled to the processor, the display configured for displaying a current status of the timer.

5. The apparatus of claim 4, wherein the rechargeable battery is further configured for powering the display.

6. The apparatus of claim 4, further comprising a speaker coupled to the housing and communicably coupled to the processor, the speaker configured for playing audio corresponding to a current status of the timer.

7. The apparatus of claim 6, wherein the rechargeable battery is further configured for powering the speaker.

8. The apparatus of claim 6, further comprising a radio frequency circuit communicably coupled to the processor and configured for receiving commands via radio frequency, wherein said commands include commands for activating and deactivating the valve.

9. The apparatus of claim 8, wherein the rechargeable battery is further configured for powering the radio frequency circuit.

10. The apparatus of claim 1, further comprising a lever communicably coupled to the valve, the lever configured for activating the valve when the lever is pressed.

11. An apparatus for applying sunscreen, the apparatus comprising:
(a) a housing;
(b) a suction cup coupled to the housing, such that the suction cup is configured to adhere to a vertical surface;
(c) one or more nozzles coupled to the housing, the one or more nozzles for dispensing sunscreen via a mist;
(d) a valve communicably coupled to the one or more nozzles, such that the valve regulates ingress of sunscreen to the one or more nozzles;
(e) a non-pressurized canister of sunscreen removably coupled to the valve via a fastener, such that sunscreen from the canister enters the valve;
(f) a pump for pumping sunscreen from the canister to the valve; and
(g) a processor communicably coupled to the valve, the processor configured for starting a timer and activating the pump and the valve for a predefined period of time after the timer expires.

12. The apparatus of claim 11, further comprising a push button coupled to the housing, the push button configured for activating the timer.

13. The apparatus of claim 12, further comprising a rechargeable battery configured for powering the processor, the valve and the pump.

14. The apparatus of claim 13, further comprising a display coupled to the housing and communicably coupled to the processor, the display configured for displaying a current status of the timer.

15. The apparatus of claim 14, wherein the rechargeable battery is further configured for powering the display.

16. The apparatus of claim 14, further comprising a speaker coupled to the housing and communicably coupled to the processor, the speaker configured for playing audio corresponding to a current status of the timer.

17. The apparatus of claim 16, wherein the rechargeable battery is further configured for powering the speaker.

18. The apparatus of claim 16, further comprising a radio frequency circuit communicably coupled to the processor and configured for receiving commands via radio frequency, wherein said commands include commands for activating and deactivating the valve.

19. The apparatus of claim 11, further comprising a lever communicably coupled to the valve, the lever configured for activating the valve when the lever is pressed.

20. An apparatus for applying sunscreen, the apparatus comprising:
(a) a housing;
(b) a suction cup coupled to the housing, such that the suction cup is configured to adhere to a vertical surface;
(c) one or more nozzles coupled to the housing, the one or more nozzles for dispensing sunscreen via a mist;
(d) a valve communicably coupled to the one or more nozzles, such that the valve regulates ingress of sunscreen to the one or more nozzles;
(e) a pressurized canister of sunscreen removably coupled to the valve via a fastener, such that sunscreen from the canister enters the valve;
(f) a radio frequency circuit communicably coupled to the processor and configured for receiving commands via radio frequency, wherein said commands include commands for activating and deactivating the valve; and
(g) a processor communicably coupled to the valve and the radio frequency circuit, the processor configured for activating and deactivating the valve responsive to said commands.
